(19) United States
(12) Patent Application Publication
(10) Pub. No.: US 2024/0315753 A1
(43) Pub. Date: Sep. 26, 2024

(54) HEMOSTATIC THERMAL SEALER

(71) Applicant: Medtronic Advanced Energy LLC, Minneapolis, MN (US)

(72) Inventors: Roger D. Greeley, Minneapolis, MN (US); Mark Guirguis, Minneapolis, MN (US)

(21) Appl. No.: 18/737,669

(22) Filed: Jun. 7, 2024

Related U.S. Application Data
(63) Continuation of application No. 16/053,161, filed on Aug. 2, 2018, now Pat. No. 12,023,082.
(60) Provisional application No. 62/568,953, filed on Oct. 6, 2017.

(51) Int. Cl. A61B 18/08; A61B 17/00; A61B 18/00; A61B 18/10

(52) U.S. Cl. CPC A61B 18/082; A61B 18/10; A61B 2017/00853; A61B 2018/00083; A61B 2018/00101; A61B 2018/00107; A61B 2018/00142; A61B 2018/0063; A61B 2018/00636; A61B 2018/00642; A61B 2018/00714; A61B 2018/00791; A61B 2018/00922; A61B 2018/087

(57) ABSTRACT

A hemostatic sealer includes a handle having a switch to activate a source of thermal 5 energy and a thermal assembly coupled to the handle. The thermal assembly includes an electrically resistive material disposed on an electrically insulative substrate. The resistive material is coupled to the switch to receive the source of thermal energy.

### RELATED APPLICATION

This application is a continuation of U.S. application Ser. No. 16/053,161 filed Aug. 2, 2018, which claims the benefit of U.S. Provisional Application No. 62/568,953 filed Oct. 6, 2017, each of which is hereby fully incorporated herein by reference.

### BACKGROUND

This disclosure relates generally to the field of medical devices, systems and methods for use in surgical procedures. More specifically, this disclosure relates to surgical devices, units, systems and methods that can provide for hemostasis or sealing of bodily tissues including bone.

The management and control of intraoperative bleeding can include the techniques of coagulation, hemostasis, or sealing of tissues and are often performed with the aid of electrodes energized from a suitable power source. Typical electrosurgical devices apply an electrical potential difference or signal between an active electrode and a return electrode on a patient's grounded body or between an active electrode and a return electrode on the device to deliver electrical energy to the area where tissue is to be affected. Electrosurgical devices pass electrical energy through tissue between the electrodes to provide coagulation to control bleeding and hemostasis to seal tissue. The electrosurgical devices are usually held by the surgeon and connected to the power source, such as an electrosurgical unit having a power generator, via cabling.

Dry-tip electrosurgical devices can adversely affect tissue and surgical procedures by desiccating or perforating tissue, causing tissue to stick to the electrodes, burning or charring tissue, and generating smoke at the surgical site. More recently, fluid-assisted electrosurgical devices have been developed that use saline to inhibit such undesirable effects as well as to control the temperature of the tissue being treated and to electrically couple the device to the tissue. Fluid-assisted electrosurgical devices have been developed which, when used in conjunction with an electrically conductive fluid such as saline, may be moved along a tissue surface without cutting the tissue to seal tissue to inhibit blood and other fluid loss during surgery.

Fluid-assisted electrosurgical devices apply radiofrequency (RF) electrical energy and electrically conductive fluid to provide for sealing of soft tissues and bone in applications of orthopedics (such as total hip arthroplasty, or THA, and total knee arthroplasty, or TKA), spinal oncology, neurosurgery, thoracic surgery, and cardiac implantable electronic devices as well as others such as general surgery within the human body. The combination of RF energy and the electrically conductive fluid permits the electrosurgical device to operate at approximately 100 degrees Celsius, which is nearly 200 degrees Celsius less than traditional electrosurgical devices. Typically, hemostasis is performed with fluid-assisted devices having electrodes in the bipolar arrangement that are referred to as bipolar sealers. By controlling bleeding, bipolar sealers have been demonstrated to reduce the incidence of hematoma and transfusions, help maintain hemoglobin levels, and reduce surgical time in a number of procedures, and may reduce the use of hemostatic agents.

### SUMMARY

This summary is provided to introduce a selection of concepts in a simplified form that are further described below in the Detailed Description.

An aspect of the present disclosure includes a surgical system, method, and surgical devices that can provide the therapeutic analog of traditional electrosurgical hemostatic sealing without dispersing or constantly dispersing fluid such as saline or deionized water at the surgical site. The use of the surgical device as a hemostatic sealer simplifies surgical preparation and procedures as it reduces the use of suction, and can eliminate hot saline at the surgical site. Further, the surgical device provides precise temperature control in various conditions and targeted treatment. The surgical device can be more ergonomic via a lighter attached cable, such as a cable without a saline tube, or a cordless handpiece.

In one aspect, the disclosure is directed to a surgical device configured as a hemostatic sealer. The hemostatic sealer includes a handle having a switch to activate a source of thermal energy and a thermal assembly coupled to the handle. The thermal assembly includes an electrically resistive material disposed on an electrically insulative substrate. The resistive material is coupled to the switch to receive the source of thermal energy.

In another aspect, the disclosure is directed to a surgical device configured as a hemostatic sealer. The surgical device includes a handle having a switch, a shaft extending distally from the handle, and a thermal assembly operably coupled to the distal end of the shaft. The thermal assembly includes a heating element and an electrically insulative substrate. The substrate provides a shape of the thermal assembly. The heating element includes an electrically resistive material electrically coupled to the switch and disposed on the substrate.

The surgical device includes a thermal assembly that provides even thermal therapy and bleeding management with precise temperature control that will interface with tissue. Activation of the thermal assembly quickly heats a heating element into the temperature range of about 80 degrees Celsius to 110 degrees Celsius, i.e., the known temperature range for good hemostasis, and will quickly reduce heat at deactivation of the thermal assembly to avoid inadvertent thermal damage to tissue, the surgical drape, or other surgical equipment. In one example, the thermal assembly heats to the selected thermal range upon one second of activation and cools to a safe temperature within one second of deactivation.

Precise temperature control is provided with two features. First, the heating assembly includes a heating element formed of a thin electrically resistive material on a thermally insulative and durable substrate. The heating element is coupled to a source of electrical energy, which is not limited to the RF range. In one example, the heating element includes a thin nickel-chrome plating in the range of 0.001 to 0.005 inches thick on a ceramic substrate. Second, the resistance or impedance of the heating element changes with temperature, and the resistance or impedance is monitored with a controller. The controller maintains the target temperature in the thermal assembly via selective operation of the source of electrical energy. In one example, the source of electrical energy and the controller can be included within a handheld surgical device.

The surface of the thermal assembly is smooth and slides along the tissue without saline. In one example, the surface of the thermal assembly includes a lubricious or non-stick coating to improve lubricity.

### BRIEF DESCRIPTION OF THE DRAWINGS

FIG. 1 is a schematic view illustrating a surgical system of the disclosure.

FIG. 2 is a schematic view illustrating an example architecture of the surgical system of FIG. 1.

FIG. 3 is a perspective view illustrating an example of a surgical device of the system of FIG. 1 including a thermal assembly.

FIG. 4 is a schematic view illustrating example features of the thermal assembly of the surgical device of FIG. 3.

FIGS. 5A and 5B are perspective views of an example heating assembly suitable for use with surgical device of FIG. 3.

FIGS. 6A and 6B are perspective views of another example heating assembly suitable for use with surgical device of FIG. 3.

### DETAILED DESCRIPTION

Throughout the description, like reference numerals and letters indicate corresponding structure throughout the several views. Also, any particular features(s) of a particular exemplary embodiment may be equally applied to any other exemplary embodiment(s) of this specification as suitable. That is, features between the various exemplary embodiments described herein are interchangeable as suitable and may not be exclusive. From the specification, it should be clear that the terms “distal” and “proximal” are made in reference to a user of the device.

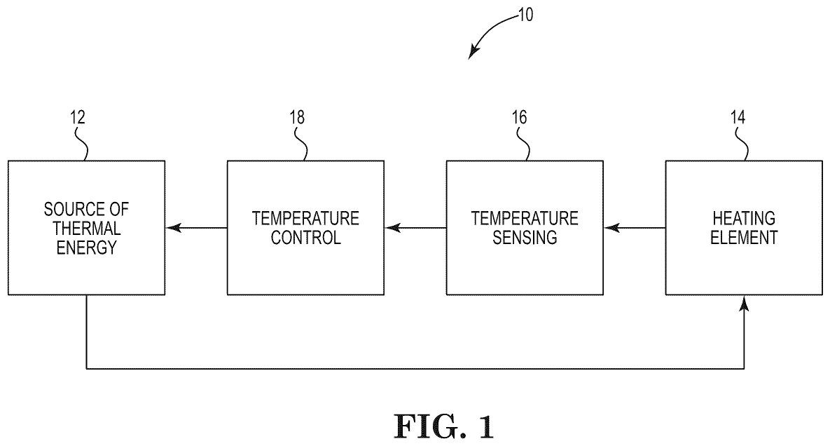

FIG. 1 illustrates a surgical system 10 that can include a handheld surgical device to deliver thermal energy to provide hemostasis or sealing of body tissues including bone without the use or fluid or without the constant dispersal of fluid. In one example, the system 10 can be included within a handheld surgical device. In still another example, the system 10 can include a selectively dispersed fluid, e.g. a fluid can be dispersed at the discretion of the operator of the system 10 rather than constantly dispersed while the device is activated.

The system 10 includes a source of thermal energy 12 coupled to a heating element 14. In one example, the source of thermal energy 12 includes a source of electrical energy electrically coupled to the heating element 14. The heating element 14 can be configured as part of heating assembly on a distal tip of a surgical device. The heating element 14 can include a resistive material that is configured to rise in temperature when an electrical current is passed through the heating element 14. The source of thermal energy 12 can be selectively activated via a switch to apply the electrical current to the heating element 14. Activation of the thermal assembly quickly heats the heating element 14 into the temperature range of about 80 degrees Celsius to 110 degrees Celsius, such as to a preselected temperature within that range. In one example, the heating element include a low thermal mass or heat capacity so that the heating clement 14 heats to the preselected temperature or temperature range upon one second of activation and cools to a safe temperature within one second of deactivation.

The system 10 includes a temperature detection mechanism 16 operably coupled to detect the temperature of the heating element 14. The temperature detection mechanism 16 can directly or inferentially determine temperature of the heating element 14. In one example, the temperature detection mechanism includes a thermocouple. In another example, the resistance or impedance of the heating element 14 changes with temperature, and the resistance or impedance of the heating element 14 is detected with the temperature detection mechanism 16. The temperature detection mechanism 16 is operably coupled to a controller 18 to monitor the temperature of the heating element 14. The controller 18 can include a processor and memory to execute a set of instructions in an application to monitor and control the temperature of the heating clement 14 with the source of thermal energy 12. In some examples, the system 10 can include a display or a data output couplable to an external monitor to provide graphical or indications of temperature or other information as determined by the controller 18.

In some examples, the system 10 may provide for a selective application of fluid if desired by a surgeon. Fluid may be provided from a fluid source that can include a bag of fluid through a drip chamber to delivery tubing and to a handheld surgical device. In one example, the fluid includes saline and can include physiologic saline such as sodium chloride (NaCl) 0.9% weight/volume solution. Saline is an electrically conductive fluid, and other suitable electrically conductive fluids can be used. In other examples, the fluid may include a nonconductive fluid, such as deionized water.

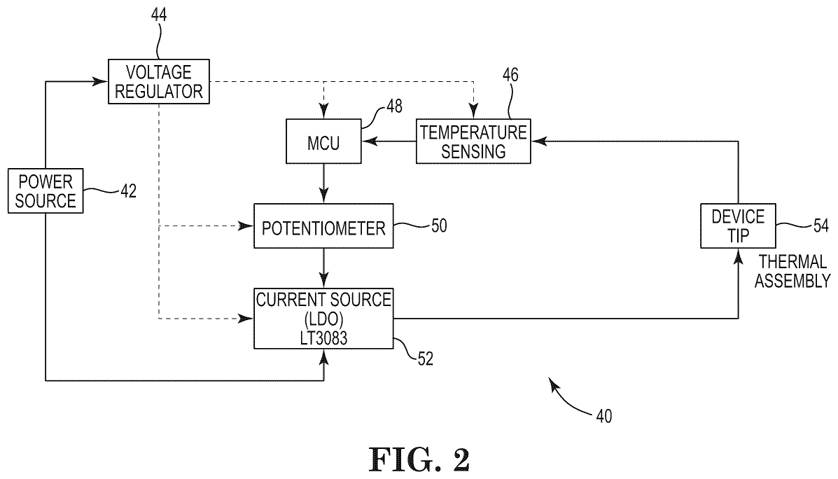

FIG. 2 illustrates an example system architecture 40, which can correspond with the surgical system 10. System architecture 40 includes a power source 42 operably coupled to a voltage regulator 44. The voltage regulator 44 can be coupled to a temperature sensing module 46, a controller such as microcontroller unit 48, a potentiometer 50 and current source 52. The current source 52 receives a signal from the voltage regulator 44 and power source 42 to provide an electrical signal to a thermal assembly 54 including a heating element. In one example, the current source 52 can include a low-dropout, or LDO, regulator, which is a direct current (DC) linear voltage regulator that can regulate an output voltage even when a supply voltage is very close to the output voltage. The electrical signal heats the thermal assembly 54 to a preselected temperature or to a temperature within a selected temperature range. The temperature sensing module 46 detects the temperature of the thermal assembly 54 and provides the microcontroller unit 48 an output of representative of the detected temperature. The microcontroller unit 48 can monitor the temperature and selectively adjust the temperature of the thermal assembly 54 such as by adjusting the potentiometer 50 to control the current source 52.

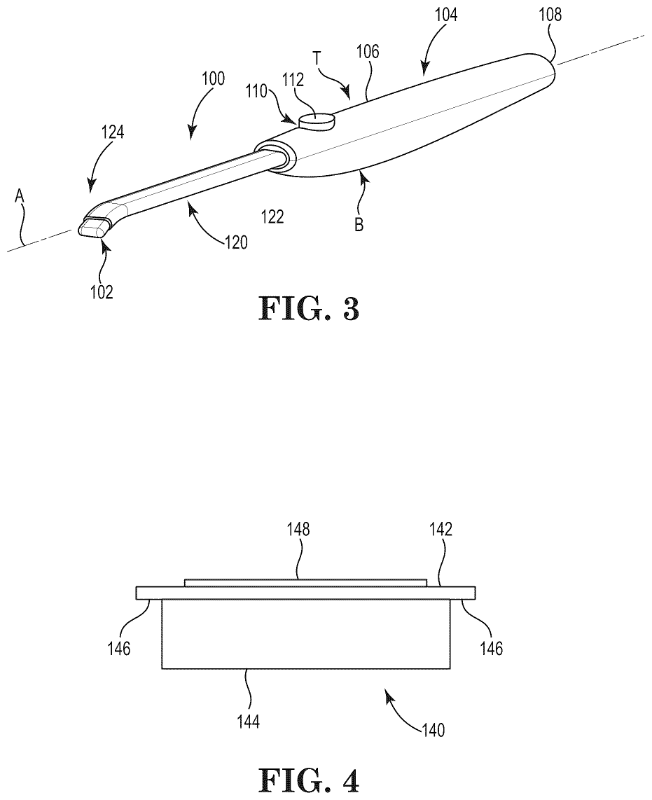

FIG. 3 illustrates an example of a surgical device 100 having thermal assembly 102 that can be used in conjunction with system 10. Thermal assembly 102 includes an exposed conductive surface configured to be electrically coupled to a source of electrical energy supplied from a power source that is not necessarily in the RF range. Thermal assembly 102 can be configured to provide for a robust electrode/tissue interface. The thermal assembly 102 may be formed to optimize hemostatic sealing of bone and tissue or coagulation without fluid, in conjunction with selected delivery of fluid, or for a particular application or anatomical geometry, or to perform other functions such as blunt dissection.

Another example of a surgical device can include a thermal assembly mounted on jaws or clamps that are movable with respect to each other. For example, jaws or clamps can selectively pinch tissue with a thermal assembly. Other examples are contemplated.

Surgical device 100 extending along longitudinal axis A includes a handpiece 104. Handpiece 104 includes a handle 106 that can include a finger grip portion with ridges (not shown) on the lower surface or bottom B of the device 100 and intended to be held in the surgeon's hand. In the illustrated example, the device 100 is cordless and includes the features of a thermal control system within the handpiece 104. The handpiece 104 includes a proximal end 108 for balance and, in one example, can include an electrical connector for electrically coupling a cable to the device 100 to supply power. Handpiece 104 may be configured to enable a user of device 100 to hold and manipulate device 100 between the thumb and index finger like a writing instrument or an electrosurgical pen. Handpiece 104 may comprise a sterilizable, rigid, electrically insulative material, such as a synthetic polymer (e.g., polycarbonate, acrylonitrile-butadiene-styrene).

The handle 106 can include an upper surface, or top T, that is opposite bottom B. A controller 110, such as a set of one or more switches 112 coupled to circuitry such as on a printed circuit board, in the example is disposed on top T and configured to be operated by the user's thumb or index finger to control one or more functions of the device 100. In the example, the switch can provide binary activation (on/off) control for each function and can be configured as a pushbutton. For example, switch 112 can be pushed to activate the thermal assembly 102 and released to deactivate the thermal assembly 102. Another switch (not shown) can be used selectively activate fluid dispersal. Other functions of the device are contemplated.

The surgical device 100 can include a probe assembly 120 extending distally from the handpiece 104. The probe assembly 120 in the example includes a shaft 122. The shaft 122, or other portions of device 100 may include one or more elements forming a subassembly to be generally one or more of rigid, bendable, fixed-length, variable-length (including telescoping or having an axially-extendable or axially-retractable length) or other configuration.

In one example, the handle 106 and shaft 122 can be formed from an electrically or thermally insulative material such as a high temperature micromolded polymer. Example insulative materials can include polytetrafluoroethylene (PTFE), polycarbonate (PC), polyoxymethylene (POM or acetal), or polyether ether ketone (PEEK).

The shaft 122 is configured to communicate a source of thermal energy to the thermal assembly 102. The shaft 122 carries one or more electrical conductors to a distal end 124 including the thermal assembly 102. Electrical pathways of the handpiece 104 and probe assembly 120 can be formed as conductive arms, wires, traces, other conductive elements, and other electrical pathways formed from electrically conductive material such as metal and may comprise stainless steel, titanium, gold, silver, platinum or any other suitable material.

In examples of the device 100 that can selectively disperse fluid, the shaft 122 includes a fluid lumen extending into the handpiece 104 for fluidly coupling to delivery tubing in a cable extending from proximal end 108. The fluid lumen includes can an outlet port disposed on or proximate the thermal assembly 102 for selectively dispersing fluid in the surgical site. In one example, fluid lumen can be included in a hypotube configured to mate with delivery tubing to supply fluid to thermal assembly 102. Hypotube can be constructed from non-conductive commonly used flexible tubing, such as polyvinyl chloride (PVC), PEEK, or a thermoplastic elastomer (TPE). In one example, the TPE is a polyether block amide (PEBA) available under the trade designation PEBAX from Arkema of Colombes, France.

FIG. 4 illustrates an example thermal assembly 140, which can correspond with thermal assembly 102. Thermal assembly 140 includes a heating element 142 disposed on a substrate 144. The heating clement 142 can include a set of tabs 146 that are configured to be communicatively coupled to a source of thermal energy. The thermal energy can be passed through the heating element 142 between the tabs 146. Various configurations and shapes of the thermal assembly 140 are contemplated, and this disclosure includes some examples of the configurations and shapes.

In one example, the source of thermal energy is an electrical current. The heating element 142 is formed of an electrically resistive material. The tabs 146 are couplable to electrical conductors in the shaft 122 and in electrical communication with the heating element 142. The electrical current is passed through the heating element 142 between the tabs 146 to heat the thermal assembly 140. In this example, electrically resistive material has a low thermal mass, or heat capacity, and can change temperature quickly depending on whether a current is applied. Additionally, the electrically resistive material can change resistance or impedance based on its temperature. In one example, the temperature of the heating element 142 can be monitored inferentially by detecting resistance or impedance of the heating element 142.

In one example, the heating element 142 is constructed from a material, such as an alloy, having a high resistivity. An example of an alloy having a high resistivity includes a nickel chrome, or nichrome, alloy. Other examples are contemplated. In one example, the heating element 142 is configured as a plating on the substrate 144. In other examples, the heating element 142 can be configured as a wire. An example thickness of a nichrome plating may be in a range of 0.001 to 0.005 inches. Other features of the heating element besides resistivity can include temperature coefficient of resistivity and corrosion resistance.

The substrate 144 in the example can be selected as having high thermal insulative properties as well as electrically insulative properties and durability. Examples of substrate 144 can include ceramics, glass, and plastics that are suitable for receiving a plating of resistive material. The substrate can be configured in a shape that is suited for the particular application of the surgical device 100. In some examples of a surgical device 100 in which fluid is selectively provided to the surgical site, the substrate may include an outlet port in fluid communication with the fluid lumen to disperse fluid from the heating assembly 102.

Portions of the thermal assembly 140 that interface with tissue are made smooth to improve lubricity so the thermal assembly 140 may slide along tissue without sticking. In one example, the surface of the thermal assembly 140 can include a lubricious coating 148 such as PTFE to further improve lubricity. The thermal assembly 140 is configured to slide smoothly along tissue without the use of saline.

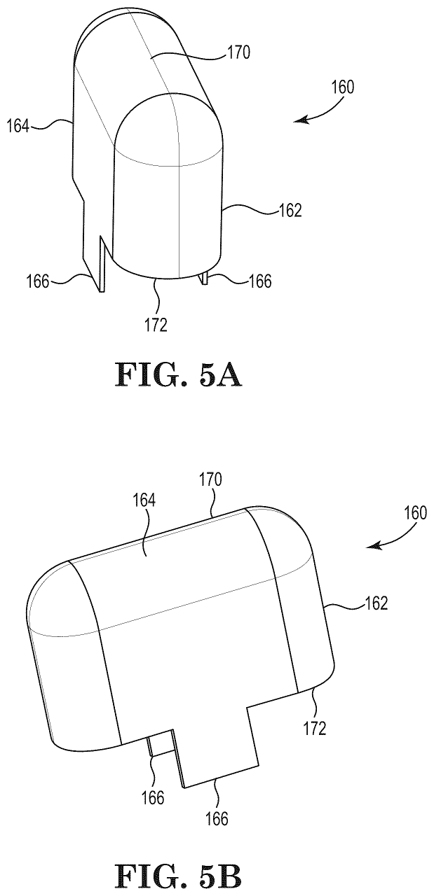

FIGS. 5A and 5B illustrate an example of a thermal assembly 160 that can correspond with thermal assemblies 140 and 102. The thermal assembly 160 includes a general shape of a blunted rectangular prism having a distal tip 170 and a proximal end 172. The thermal assembly 160 includes a blunted substrate 162 formed from a thermally insulative, electrically insulative, and durable material such as a ceramic, glass, plastic, or other material. A heating element 164 is plated over the distal tip 170 of the substrate 162 and can be made of a resistive material such as thin nichrome. The heating element 164 can be electrically coupled to an electrical signal, such as a current source, via tabs 166 on opposite sides of the substrate 162 proximate the proximal end 172.

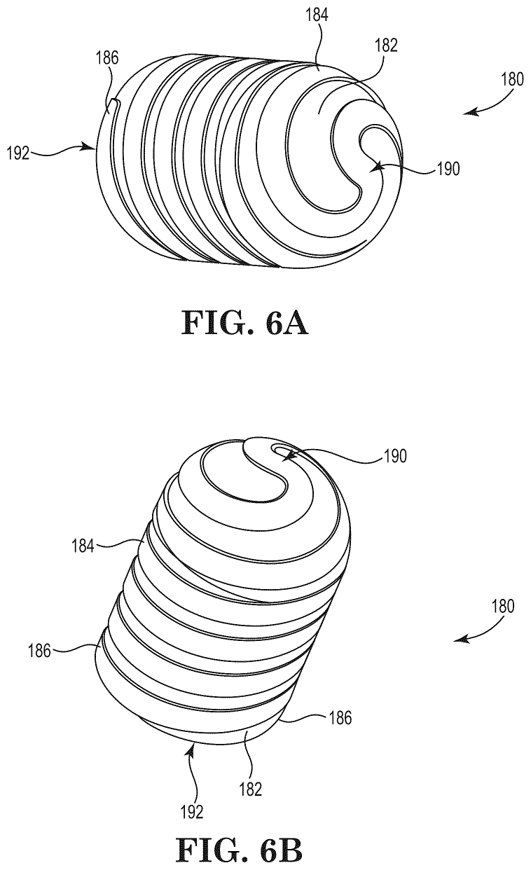

FIGS. 6A and 6B illustrate another example of a thermal assembly 180 that can correspond with thermal assemblies 140 and 102. The thermal assembly 180 includes a general shape of a blunted ogive or paraboloid having a distal tip 190 and a generally circular proximal end 192. The thermal assembly 180 includes a blunted substrate 182 formed from a thermally insulative, electrically insulative, and durable material such as a ceramic, glass, plastic, or other material. A heating element 184 is plated as a single electrical path configured as double helical trace over the distal tip 190 of the substrate 182 and can be made of a resistive material such as thin nichrome. The heating element 184 can be electrically coupled to an electrical signal, such as a current source, via tabs 186 on opposite sides of the substrate 182 proximate the proximal end 192. The tabs 186 in the example generally correspond with ends of the helical trace.

In one example, thermal assemblies 160, 180 can be attached to a shaft, such as shaft 122, and heating elements 164, 184 can be electrically coupled to electrical pathways within the shaft 122. Thermal assemblies 160, 180 can include lubricious coatings (not shown) such as PTFE over the heating elements 164, 184 to improve lubricity. Lubricious coatings may also cover the substrates 162, 182.

Although the present disclosure has been described with reference to preferred embodiments. workers skilled in the art will recognize that changes can be made in form and detail without departing from the spirit and scope of the present disclosure.

What is claimed is:

1. An surgical device, comprising:
a handle including a switch;
a shaft extending distally from the handle, the shaft including a distal end; and
a thermal assembly operably coupled to the distal end of the shaft, the thermal assembly including a heating element and an electrically insulative substrate wherein the substrate provides a shape of the thermal assembly and the heating element includes an electrically resistive material electrically coupled to the switch and disposed on the substrate.

2. The surgical device of claim 1 wherein the thermal assembly is configured to provide hemostatic sealing of tissue without a fluid.

3. The surgical device of claim 1 including a controller to monitor and adjust the temperature of the thermal assembly.

4. The surgical device of claim 3 wherein the heating element provides a temperature in the temperature range of 80 degree Celsius and 110 degrees Celsius when activated.

5. The surgical device of claim 1 wherein the resistive material is a nickel chrome alloy.

6. The surgical device of claim 1 wherein the resistive material if formed as a plated trace on the substrate.

7. The surgical device of claim 6 wherein the plated trace is in a helical configuration on the substrate.

8. The surgical device of claim 1 wherein the resistive material is 0.001 to 0.005 inches thick on the substrate.

9. The surgical device of claim 1 wherein the substrate is a formed of a thermal insulator.

10. The surgical device of claim 9 wherein the thermal insulator is ceramic.

11. The surgical device of claim 1 wherein the thermal assembly includes a lubricious coating.

12. The surgical device of claim 11 wherein the lubricious coating includes PTFE.

13. A hemostatic sealer, comprising:
a handle having a switch to activate a source of thermal energy; and
a thermal assembly coupled to the handle, the thermal assembly including an electrically resistive material disposed on an electrically insulative substrate, the resistive material operably coupled to the switch to receive the source of thermal energy.

14. The hemostatic sealer of claim 13 operably couplable to a source of electrical energy to provide the thermal energy, wherein the resistive material provides an electrical path for the source of electrical energy without a fluid.

15. The hemostatic sealer of claim 14 wherein the source of electrical energy is included in the handle.
